(19) United States
(12) Patent Application Publication
(10) Pub. No.: US 2009/0136926 A1
(43) Pub. Date: May 28, 2009

(54) Device and method for standardizing nucleic acid concentrations

(76) Inventors: Ralf Himmelreich, Langenfeld (DE); Christoph Erbacher, Haan (DE); Dirk Loffert, Dusseldorf (DE)

Correspondence Address: MCDERMOTT WILL & EMERY LLP, 600 13TH STREET, N.W., WASHINGTON, DC, 20005-3096, US

(21) Appl. No.: 11/921,415

(22) PCT Filed: May 9, 2006
(86) PCT No.: PCT/EP2006/062153

§ 371 Date: Oct. 7, 2008

(30) Foreign Application Priority Data
May 30, 2005 (DE) 10 2005 025 080.7

(51) Int. Cl. C12Q 1/68

(52) U.S. Cl. 435/6

(57) ABSTRACT

The present invention relates to a device and a method for normalising nucleic acid concentrations, preferably for normalising nucleic acid concentrations in enzymatic nucleic acid amplification and modification methods.

The present invention relates to a device and a method for normalising nucleic acid concentrations, preferably for normalising nucleic acid concentrations in enzymatic nucleic acid amplification and modification methods.

A known problem in the technical field to which the invention relates is that nucleic acid preparations exhibit variations in the final concentration of cleaned nucleic acids depending on the starting material and/or the method of cleaning. Therefore, in nucleic acid amplification and modification methods, the cleaned nucleic acids are usually quantified and if necessary adjusted to a standardised concentration, that is to say the samples are normalised, before the reaction. Only then is a meaningful quantification and comparison of the samples by means of quantitative PCR or other verification procedures possible. A further possible quantification method is the simultaneous amplification of a standard. These standards constitute defined nucleic acid sequences and serve as markers. The origin of the sequence can even stem from a different organism. The task of this standard is to display the reaction conditions and to enable the data to be analysed.

Alternative preparation methods are known from the prior art (for example the ‘IQ System’ from Promega, Madison, USA or ‘Charge Switch gDNA Normalized Buccal Cell Kit’ from Invitrogen, Carlsbad, USA), which allow uniform concentrations of nucleic acids (in particular genomic DNA) to be prepared from different starting materials. However, it has been shown in practice that these technologies still exhibit an extremely strong variation in the yields (standard deviation from the mean >>10%), and this therefore does not result in a uniform concentration of nucleic acids.

Currently no preparation method is known from the prior art for which a quantification and normalisation is not necessary if the intended downstream reaction requires such. There is therefore a strong demand in professional circles for a method, which enables simple and accurate normalisation.

The disadvantages known from the prior art are solved by the present invention. This enables effective, precise, simple and rapid normalisation of the cleaned nucleic acids without quantification. This is achieved by means of modified consumables.

The present invention provides a device for the normalisation of nucleic acid concentrations. The device according to the invention has a surface, the form of which is modified so that it has a defined nucleic-acid-binding capacity. The binding capacity is limited by the extent of the surface in or on the device and its chemical functionalisation. Free nucleic acids in aqueous solution are bound by the modified surfaces until saturation; the surface is accordingly unable to absorb an excess of nucleic acid.

Examples of devices within the meaning of the invention, that is having a modified surface with defined nucleic-acid-binding capacity, can be PCR vessels (e.g. Eppendorf tubes), multi-well plates (e.g. 96-well plates or 384-well plates) and also films or so-called ‘dipsticks’. ‘Dipsticks’ within the meaning of the invention are understood to mean preferably rods made of glass or plastic with a surface modified according to the invention, which are immersed in a solution containing nucleic acid, and the nucleic acids are able to bind to the surface. Forms, materials and surface structure of such dipsticks are sufficiently known to the person skilled in the art. Basically, the whole surface, which is in contact with the solution containing nucleic acid, (for example the whole of the inside of a PCR tube) can be modified or, alternatively, defined areas of the surface can be modified.

The binding capacity of the modified surface for nucleic acids is based on a chemical functionalisation, which allows a reversible binding of the nucleic acids. For example, the nucleic acids can bind by means of nucleic acids or nucleic acid analogues immobilised on the surface. These are preferably present in the form of oligonucleotides. Within the meaning of the invention, immobilised nucleic acids or nucleic acid analogues are understood to mean DNA, RNA, DNA-RNA hybrids, PNA and locked nucleic acids. Further nucleic acid analogues, which can undergo a reversible binding with nucleic acids, are well known to the person skilled in the art and can also be used in the invention. The sequence of the immobilised nucleic acids or nucleic acid analogues can be random and therefore give rise to an unspecified binding of the nucleic acids. However they can also have an oligoT sequence for the specific binding of eukaryotic mRNA to the PolyA tail or alternatively a defined sequence for the sequence-specific binding of nucleic acids. The sequence can also be chosen so that a triple helix is formed with double-stranded nucleic acid to be bound.

The binding capacity of the modified surface for nucleic acids can furthermore be based on ironic layers, for example coatings with anion or cation exchange material. Typical anion exchange materials are sufficiently known in professional circles and, under certain conditions, enable the reversible and unspecific binding of nucleic acids to the surface. The binding of the nucleic acids is reversible and can be initiated for example by heat, e.g. by heating the sample in a PCR. Typical cation exchange materials have surfaces for example, which carry sulphonate, carboxyl and/or phosphate groups on the surface. The reversible binding of nucleic acids to cation exchangers is sufficiently known in professional circles.

Hydrophobic layers can also produce the binding capacity of the modified surface for nucleic acids. Such a layer consists of polypropylene for example. Many commercially available reaction vessels (e.g. Eppendorf tubes) are made of polypropylene. In order to create a defined hydrophobic surface, parts of the surface of such a device made from polypropylene can be hydrophilised. In order to reversibly bind nucleic acids from a solution to a hydrophobic surface, this must first be mixed with a molecule, which has a positive charge and a hydrophobic residue in order to enable an interaction, for example with diethylammonium ions. Alternative methods for the reversible binding of nucleic acids to hydrophobic surfaces are well known to the person skilled in the art.

As well as the first modified surface with binding capacity for nucleic acids described above, the devices according to the invention can optionally also have a second such modified surface. As the binding capacity of the first modified surface is limited by the extent of the surface and its chemical functionalisation, a constant quantity of nucleic acid always binds to this surface. Excess nucleic acid can be removed from the device (for example by draining the sample, rinsing the device, etc.). If, however, the amount of nucleic acid in the sample is too small, so that the binding capacity of the first modified surface exceeds the available amount of nucleic acid, then it will no longer be possible to carry out an exact normalisation of the amount of nucleic acid for subsequent reactions with the first modified surface. In this case, it would only be possible to demonstrate very small amounts of nucleic acids in a quantitative PCR for example. In such a case, it cannot be determined whether this is caused by a poor sample quality, for example, and/or too small a quantity of starting material and/or even an incorrectly made-up reaction mixture for example. The reaction is checked by the optional second modified surface, which likewise has a defined binding capacity for nucleic acids. In contrast to the first modified surface however, a defined amount of nucleic acid, that is to say DNA or RNA, of a defined sequence is already immobilised on the second modified surface. The sequence and the length of these nucleic acids is determined such that they are suitable for the subsequent reaction, for example a PCR or an RT-PCR. In a PCR or RT-PCR, these nucleic acids are amplified by means of suitable primers. The nucleic acids already bound here serve as a standard in a subsequent reaction. An exact qualification of the nucleic acid from the sample is therefore possible and, at the same time, the standard is used for checking the efficiency of the subsequent reaction. The binding of the nucleic acids to the second modified surface can take place covalently, for example, the amplification of the standard then being carried out by means of a fixed phase PCR, for example. However, the nucleic acids can also be bound to the second modified surface non-covalently, but must only be released from this at the beginning of the subsequent reaction and not, for example, when filling the device with the sample or when washing the device. As defined by the present invention, the nucleic acids can be bound to a second modified surface, which has an anion or cation exchanger surface, or which has a hydrophobic surface, or alternatively the binding of the nucleic acids can take place by means of nucleic acids bound to the surface. The options are basically the same as those for binding nucleic acids from the sample to the first modified surface (see above). Furthermore, a prerequisite for the binding of nucleic acids to the second modified surface is that the second modified surface is completely saturated with the nucleic acid, which is being used as a standard, in order not to cause variations of the binding capacity for the nucleic acid originating from the sample. In an alternative embodiment of the invention, nucleic acids being used as a standard can also be bound to surfaces, which are not added to the device according to the invention until the beginning of the subsequent reaction, for example by means of plastic or glass particles, e.g. in the form of wafers or balls etc., on which a defined quantity of nucleic acid being used as a standard is applied, e.g. by means of one of the methods listed above. The person skilled in the art is familiar with the size, form and type of construction of such devices. Alternatively, a nucleic acid being used as a standard can also be added to the sample before the subsequent reaction by means of a pipette, wherein however the first two options mentioned rule out any inaccuracies due to pipetting.

The modification of the surface of the device can take place in different ways depending on the manner in which the nucleic acids are to be bound to the device. Some examples of suitable methods are listed below. However, any other methods, which seem appropriate to the person skilled in the art, can be used.

Plasma method (low-pressure plasmas, but atmospheric pressure plasmas are preferred): Pulsed barrier discharges operated at atmospheric pressure have been used for the surface activation of polymers for subsequent printing, pasting or painting for a long time. A new area of application for this discharge is opened up by its use for plasma-assisted coating and cleaning processes at atmospheric pressure. By feeding compounds, such as for example glycidyl methacrylate, acrylic acid, fluorinated hydrocarbons, silicon-organic compounds, N₂ or O₂ and other compounds, which are gaseous under the applied conditions, into the discharge area, layers can be deposited on substrates or, in the case of N₂ und O₂, covalent modifications of plastics, for example, with amino groups or carboxy and hydroxyl groups, can be achieved. Due to the possibilities of maintaining this discharge in very small volumes, new perspectives open up for the structured modification of the chemical and physical properties of surfaces and for the treatment of inner surfaces of devices, for example microfluidic components or small sample vessels such as PCR vessels for example. The method can be used to produce nucleic-acid-binding areas in a PCR vessel in an objective and regionally selectively manner in order to reversibly bind a defined quantity of nucleic acids.

### CVD Method (Chemical Vapour Deposition)

This method is often used in combination with plasma methods. The device, for example a PCR vessel, is exposed to a vapour, which contains one or more decomposing or reactive chemical starting compounds. The decomposition reaction takes place on the surface of the device and the products of decomposition are deposited, as a result of which the surface is coated. The method can be used to produce nucleic-acid-binding areas in a device, such as a PCR vessel for example, in an objective and regionally selectively manner in order to reversibly bind a defined quantity of nucleic acids.

### PVD Method (Physical Vapour Deposition)

This method is characterised in that the deposited material does not undergo any chemical change and is physically deposited as it was in the gaseous phase. The method can be used to produce nucleic-acid-binding areas in a device, for example in a PCR vessel, in an objective and regionally selectively manner in order to reversibly bind a defined quantity of nucleic acids.

Known wet chemical methods can also be used to produce nucleic-acid-binding surfaces in a device such as a PCR vessel, e.g. by oxidation of the surface of the PCR vessel by means of strongly oxidising acids, so that carboxyl groups, aldehyde groups and/or hydroxyl groups are formed, in order to obtain nucleic-acid-binding areas in the device after further wet chemical process steps and to reversibly bind a defined quantity of nucleic acid. Other wet chemical methods can also be used, for example methods in which affinity ligands are covalently immobilised in a plasma-activated or chemically functionalised device, such as a PCR vessel, in order to bind nucleic acids. For example carbodiimides can be used for coupling the affinity ligands. By way of example, EDC (1-ethyl-3-(3-dimethylaminopropyl)carbodiimide-hydrochloride) can be used to activate a carboxylated surface by means of a so-called reactive ester intermediate stage. The ester formed on the surface, e.g. hydroxybenzotriazole ester or N-hydroxysuccinimide ester, is then converted with an amino functionalised affinity ligand in a second step. Biotin, streptavidin, amino functionalised oligonucleotides, PNA, nucleic-acid-binding proteins, antibodies or similar affinity ligands, which appear helpful to the person skilled in the art, are suitable as affinity ligands.

Nucleic-acid-binding synthetic surfaces can also be produced by using the nucleic-acid-binding additives in an injection moulding process for manufacturing the device, for example a PCR vessel. Preferred here are polymer additives, which contain ionic groups such as ammonium or phosphonium groups, carboxyl groups, sulphonate or phosphate residues. These polymer additives lead to a change in the surface charge of the injection-moulded device. Under suitable binding conditions, nucleic acids can then be reversibly immobilised on the surface of the device according to the invention. In this way, the required nucleic-acid-binding properties can be obtained right at the manufacturing stage of the device. Starting from this nucleic-acid-binding surface, it is also possible by means of regionally selective plasma mask technology to create patterns in the surface of the device, enabling nucleic-acid-binding and non-binding areas to be obtained.

Graft polymerisation methods are also suitable for modifying the surface of a device according to the invention. With these methods, a thin polymer film, which is covalently bonded to the surface of the vessel, is produced by radical polymerisation. In doing so, the radical polymerisation can be photo initiated or also thermally initiated or initiated by energy-rich radiation. Suitable monomers are provided in the device, which either have ionic groups or reactive groups such as epoxy groups, for example, for integrating further chemical functionalities, which are suitable for reversibly binding nucleic acids.

Coatings of the device according to the invention, which are capable of forming non-covalent bonds by means of “dip-coating” with a nucleic-acid-binding polymer, are also suitable. Preferred polymers in this regard are ionic polymers with ammonium, phosphonium, sulphonium, carboxy, sulphonate or phosphate groups. At the same time, the polymer can also have one or more of the functional groups mentioned. With this method, the device is brought into contact with the polymer, which is intended for the coating and is present in a certain concentration dissolved in a solvent. After removing the solvent, such as water or organic solvent, a thin film of the coating polymer remains on the surface of the device, which enables reversible nucleic acid binding. Such coating methods are familiar to the relevant person skilled in the art.

Furthermore, the present invention provides a method for normalising nucleic acid concentrations, preferably for normalising nucleic acid concentrations in enzymatic nucleic acid amplification and modification methods. The device according to the invention is used with this method. Basically, the method according to the invention includes the following steps:
a) Bringing the device according to the invention into contact with a sample containing nucleic acids,
b) Incubating under conditions and for a period until the nucleic-acid-binding capacity of the modified surface of the device according to the invention is exhausted,
c) Discarding the remaining sample,
d) Optional washing of the device according to the invention.

Within the meaning of the invention, samples containing nucleic acid are solutions, which already contain cleaned nucleic acids. These nucleic acids can be DNA or RNA, for example plasmides, PCR products or a preparation of DNA, for example genomic DNA, or RNA, or of total nucleic acid from a biological sample. Within the meaning of the invention, nucleic acids in solutions are cleaned when they are not present in a ‘crude lysate’ of a biological sample.

The incubation period and the respective binding conditions in order to bind the nucleic acid to the modified surface depend on the type of surface modification used in each case, but are part of the prior art and are obvious to the person skilled in the art or can be determined by the simplest routine work.

After the nucleic acids have bonded to the modified surface, the rest of the sample can be discarded, or the sample can be removed from the surface or the surface from the sample. If the device according to the invention is provided in the form of a vessel, for example, e.g. an Eppendorf tube or multi-well plate, the sample can simply be drained or sucked away or removed by pipette. If the device according to the invention is provided in the form of a dipstick, this can simply be removed from the sample.

The surface, to which the nucleic acid from the sample is now bound, can optionally be washed. A liquid is used for this purpose, for example a suitable buffer, with which contaminants can be washed away although the nucleic acids remain bound. Such liquids are part of the prior art and are therefore well known to the person skilled in the art. The liquid used for washing is subsequently discarded.

The device with the bound nucleic acids can then be used in a subsequent reaction, for example a PCR or RT-PCR. In the case where the device according to the invention is a vessel, such as an Eppendorf tube or multi-well plate, the solution required for the subsequent reaction is placed in the vessel. If the subsequent reaction is a PCR, for example, then all the components necessary for the PCR (with the exception of the nucleic acid to be amplified) are put into the device according to the invention in the form of a PCR vessel and the reaction is started. By heating the solution to start the PCR, the nucleic acids are released from the reversible bond and the reaction can proceed with a defined quantity of nucleic acids. In the case where the device according to the invention is a dipstick or an equivalent device, the part of the device to which the nucleic acids are bound is put into an appropriate device in which the subsequent reaction proceeds. If the subsequent reaction is a PCR, for example, then the dipstick or the part of the dipstick is simply put into a PCR tube already containing all components for the PCR (with the exception of the nucleic acid to be amplified). The rest of the process is as described above. If the device according to the invention is used in the form of a dipstick, then it has been shown to be advantageous that the dipstick has a deliberate breakpoint, by means of which the part binding the nucleic acids can easily be separated from the rest of the device. This can remain in the reaction vessel during the subsequent reaction.

Methods, which require a normalisation of the nucleic acid quantities, or in which such a normalisation is shown to be very advantageous, are sufficiently known to the person skilled in the art and can be carried out after the method according to the invention.

Alternatively, the device used in the method according to the invention can also have a second modified surface with defined nucleic-acid-binding capacity, wherein nucleic acids being used as a standard are already immobilised on this modified surface. This embodiment of the invention is described in more detail above. In this case, as already mentioned above, it is of decisive importance that the nucleic acids being used as a standard are only released from this modified surface at the beginning of the subsequent reaction (e.g. PCR, RT-PCR, etc.), e.g. by the initial heating of the solution in a PCR reaction, and that this second modified surface is completely saturated with the nucleic acid being used as a standard so that it does not exhibit a residual binding capacity for the nucleic acid present in the sample, and thus make it no longer possible to carry out an accurate normalisation.

Furthermore, the present invention relates to kits for carrying out the method according to the invention and containing at least one device according to the invention.

1. Device for the normalization of nucleic acid concentrations having a modified surface with defined nucleic-acid-binding capacity.

2. Use of a device according to claim 1 in a method for the normalization of nucleic acid concentrations.

3. Kit for carrying out a method according to claim 2 containing a device.
